(19) United States
(12) Patent Application Publication
(10) Pub. No.: US 2026/0262982 A1
(43) Pub. Date: Sep. 10, 2026

(54) METHOD, SYSTEM AND DEVICE FOR ANALYZING THE COMFORT LEVEL OF HIGH-SPEED ELEVATOR PASSENGERS BASED ON EEG

(71) Applicants: China Jiliang University, Hangzhou (CN); Zhejiang University of Technology, Hangzhou (CN)

(72) Inventors: Gang Xiao, Hangzhou (CN); Xiaoru Yu, Hangzhou (CN); Zhongcheng Xiao, Hangzhou (CN); Jiawei Lu, Hangzhou (CN); Ren Li, Hangzhou (CN); Zhenbo Cheng, Hangzhou (CN)

(21) Appl. No.: 19/403,325

(22) Filed: Nov. 28, 2025

(30) Foreign Application Priority Data
Mar. 5, 2025 (CN) 202510254283.0

(51) Int. Cl. A61B 5/16; A61B 5/00; A61B 5/31; A61B 5/374; A61B 5/377; G01P 15/18

(52) U.S. Cl. CPC A61B 5/165; A61B 5/31; A61B 5/374; A61B 5/377; A61B 5/6803; G01P 15/18; A61B 2503/12

(57) ABSTRACT

The present invention provides a method, system and device for analyzing the comfort level of high-speed elevator passengers based on EEG, and it can simultaneously collect and store acceleration data of elevator cars and EEG data of passengers; intercept EEG data and acceleration data based on the operating state of the elevator; preprocess the above data; form operating index and EEG index based on the feature extraction of the above data; ultimately form operating index curve and EEG index curve by recording the above index, to provide tools for correlation analysis between elevator operation and passengers' feelings, so that the evaluation of the comfort level of high-speed elevator passengers is more objective and accurate.

### CROSS REFERENCE TO RELATED APPLICATIONS

The present application claims the benefit of Chinese Patent Application No. 202510254283.0 filed on Mar. 5, 2025, the contents of which are incorporated herein by reference in their entirety.

### TECHNICAL FIELD

The present invention relates to the field of analysis on comfort level of high-speed elevator passengers, and brain science, especially providing a method, system and device for analyzing the comfort level of high-speed elevator passengers based on EEG.

### BACKGROUND ART

Elevators as the crucial vertical transportation tools have become an important component of modern urban infrastructure. The comfort level of passengers is an important index for evaluating the performance of high-speed elevators, used to assess the experience of elevator passengers. The current measurement on the comfort level of passengers mainly relies on subjective scale method that quantifies the operating data of elevator cars through the acceleration sensor and builds the comfort level evaluation system. The existing methods generally cause evaluation bias due to factors such as perception difference and memory error among different populations.

### SUMMARY OF THE INVENTION

The present invention provides a method, system and device for analyzing the comfort level of high-speed elevator passengers based on EEG to overcome the shortcomings of existing technologies. It aims to provide a new objective analysis tool, to avoid the evaluation bias caused by the uncertainty of the subjective assessment in the current evaluation on the comfort level of transportation system passengers, and improve the reliability and effectiveness of the analysis on the comfort level of passengers.

In order to achieve the above purpose, the present invention provides a method for analyzing the comfort level of high-speed elevator passengers based on EEG. It comprises the steps of:
S1: simultaneously collecting and storing human EEG signals of high-speed elevator passengers and operating signals of high-speed elevator cars;
S2: judging the changes in operating state of high-speed elevators based on operating signals of elevator cars; intercepting the human EEG signals and the operating signals of elevator cars during the changes in operating state, to obtain human EEG data and elevator car operating data;
S3: preprocessing human EEG data and elevator car operating data;
S4: conducting feature extraction for human EEG data and elevator car operating data, and forming human EEG index and elevator car operating index after analysis, wherein the step of conducting feature extraction for human EEG data includes the power spectrum analysis on intercepted EEG signals and the effective connection analysis on EEG signals;
S5: forming human EEG index curve and elevator car operating index curve by recording and storing human EEG index and the elevator car operating index, wherein the index curve is the quantitative trajectory of single index mapped in two-dimensional Cartesian coordinate system.

Preferably, the preprocessing step includes:
adopting a filter to remove the noise from the human EEG data and elevator car operating data;
resampling the human EEG data and elevator car operating data;
using the independent component analysis algorithm to remove the interference signals in the human EEG data.

Preferably, in S4, the step of conducting power spectrum analysis on intercepted EEG signals includes: using the Welch algorithm to calculate the power spectrum density of each data segment in different EEG frequency bands as an EEG power spectrum index.

Preferably, in S4, the specific step of conducting the effective connection analysis on EEG signals includes: obtaining the direct directed transfer function matrix of each data segment; calculating the stacking mean of the direct directed transfer function matrix for all data segments, dividing it into different frequency bands, and getting the mean of the direct directed transfer function for different connection directions in each frequency band as an EEG effective connection index.

The present invention also provides a system for analyzing the comfort level of high-speed elevator passengers based on EEG, including:
an acquisition module, used to collect the human EEG signals of passengers and the operating signals of high-speed elevator cars;
a trigger module, coupled with the elevator car operating data acquisition module, configured to judge the changes in operating state of high-speed elevator cars based on the elevator car operating signals, and output activation signals to data analysis module during the changes in operating state;
a data analysis module, configured to receive the activation signals, the human EEG signals and the elevator car operating signals; intercept and record the human EEG signals and the elevator car operating signals based on the activation signals, to obtain human EEG data and elevator car operating data; perform time synchronization, data preprocessing and feature extraction analysis for the human EEG data and elevator car operating data, to output EEG indexes and elevator car operating indexes; conduct power spectrum analysis on intercepted EEG signals by using the Welch algorithm at the time of carrying out feature analysis on human EEG data to obtain the power spectrum index of EEG data, and implement the effective connection analysis on EEG signals through the direct directed transfer function to get the effective connection index of EEG data.

Preferably, when the trigger module is used to judge the changes in operating state of high-speed elevator cars, the process includes:
setting the threshold value according to the historical data of the elevator car operating signals to judge if the elevator car operating signals exceed the threshold range, outputting activation signals when the judgement result is “yes”, and stopping outputting activation signals when the judgement result is “no”.

Preferably, elevator car operating signals include acceleration signals, speed signals, and attitude signals; the elevator car operating index includes the operating data's time domain feature, frequency domain feature, and information entropy; the EEG index includes the EEG data's time domain feature, frequency domain feature, information entropy and effective connection.

The present invention also provides a device for analyzing the comfort level of high-speed elevator passengers based on EEG that uses the method for analyzing the comfort level of high-speed elevator passengers based on EEG for analysis, including:
an EEG sensor, an operation sensor, an intelligent terminal, an uninterruptible power supply, a tool kit, and a mobile collection cart; the EEG sensor includes an EEG electrode cap and an EEG signal amplifier; the operation sensor includes the acceleration sensor and operation sensor host; the mobile collection cart includes an equipment layer, a tool layer, and an operation layer from top to bottom, and these layers are rigidly connected to each other through four sets of fixed rods; the equipment layer includes a storage chassis and a universal wheel connected to the bottom of the storage chassis, used to place the uninterruptible power supply; the tool layer is used to place the acceleration sensor, the EEG electrode cap, and the tool kit; the operation layer includes an operation table, wherein a handrail is set up on one side of the operation table, and one side of the bottom of the operation table is detachably connected to the EEG signal amplifier and the operation sensor host; the operation layer is used to place the intelligent terminal; the EEG sensor, the operation sensor, the uninterruptible power supply, and the intelligent terminal are electrically connected to each other;
during the analysis process, the human EEG data are collected through the EEG sensor, and the elevator car operating data are acquired through the operation sensor; the collected data are input into an intelligent terminal to judge the changes in the operating state of the elevator car, preprocess human EEG data and elevator car operating data, perform feature extraction analysis, and ultimately output human EEG index and elevator car operating index.

The present invention also provides an intelligent terminal, including:
at least a processor, and a machine-readable storage medium communicating and connecting with at least one processor; the machine-readable storage medium has computer instructions executed by at least one processor; the computer instructions are used to implement the method and system for analyzing the comfort level of high-speed elevator passengers based on EEG.

Compared with the existing technologies, the beneficial effect of the present invention is as follows:

The present invention is based on neuroscience to implement an analysis method independent of subjective feedback from passengers, avoiding the influence caused by oral communication. The present invention uses the EEG signals to analyze the comfort level of passengers during transportation, and the EEG signals have higher time resolution compared with peripheral electrophysiological analysis techniques such as ECG and GSR, and are suitable for capturing short-term neural and cognitive events in high-speed elevators; in addition, the EEG signals are directly related to advanced cognitive functions like comfort level, and peripheral physiology such as ECG and GSR is only indirectly related to advanced neural activity. Based on this, analyzing the comfort level of elevator passengers through EEG signals has higher objectivity and reliability.

### BRIEF DESCRIPTION OF THE DRAWINGS

FIG. 1 is the flowchart of the method for analyzing the comfort level of high-speed elevator passengers based on EEG provided in the present invention.

FIG. 2 is the frame diagram of the system for analyzing the comfort level of high-speed elevator passengers based on EEG provided in the present invention.

FIG. 3 is the flowchart of the system for analyzing the comfort level of high-speed elevator passengers based on EEG provided in the present invention.

FIG. 4 is the schematic diagram of the connection positions of human EEG electrodes.

FIG. 5 is the operating acceleration diagram of upward and downward high-speed elevators.

FIG. 6 is the schematic diagram of the device for analyzing the comfort level of high-speed elevator passengers based on EEG provided in the present invention.

FIG. 7 is the use diagram of the device for analyzing the comfort level of high-speed elevator passengers based on EEG provided in the present invention.

### DETAILED DESCRIPTION

The implementation mode of the present invention is explained through specific embodiments. Technicians in this field can easily understand other advantages and effects of the present invention according to the content disclosed in the specifications. The present invention can be implemented or applied through different specific implementation modes. The details in the specifications can be modified or changed based on different perspectives and applications, without departing from the spirit of the present invention. It has to be noted that, in the absence of conflict, the following embodiments and their features can be combined with each other.

It is necessary to note that, the illustrations provided in the following embodiments only illustrate the basic concept of the present invention in a schematic manner. Therefore, the illustrations only show the components related to the present invention, and are not drawn according to the actual number, shape and size of the components during implementation. In practice, the shape, quantity and proportion of each component can be arbitrarily changed, and the layout of the components may be more complicated.

The comfort level of passengers stems from human perception, wherein the nervous system plays a major regulatory role. The neural pathways of the system are widely distributed in the brainstem, cerebellum and cerebral cortex, triggering physiological responses by integrating perceptual information. Based on this, by detecting the electrophysiological signals of relevant brain regions and their parameters, it is possible to analyze the comfort level of high-speed elevator passengers.

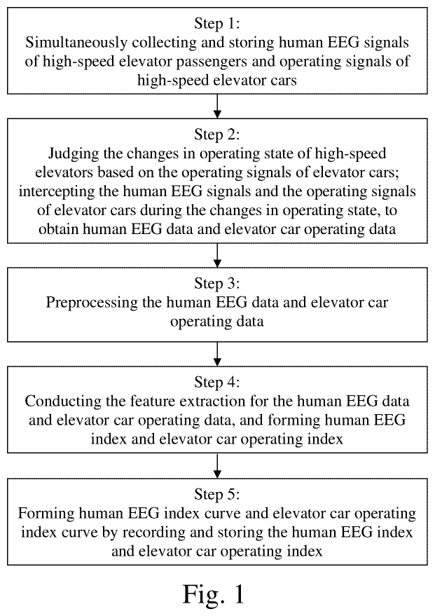

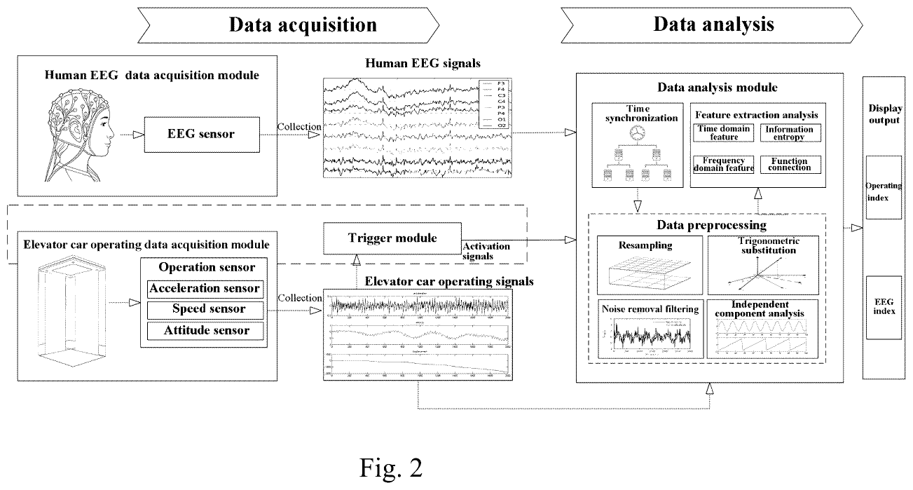

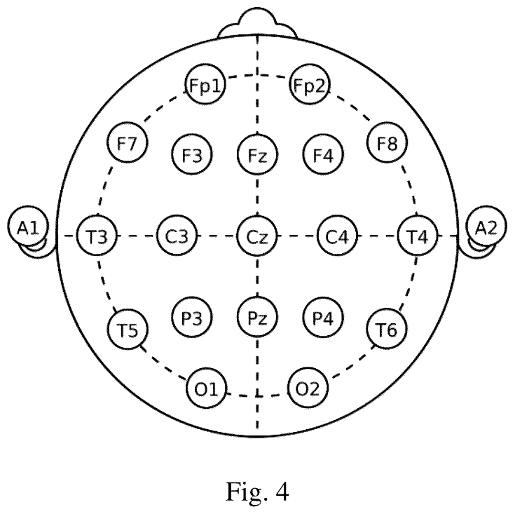

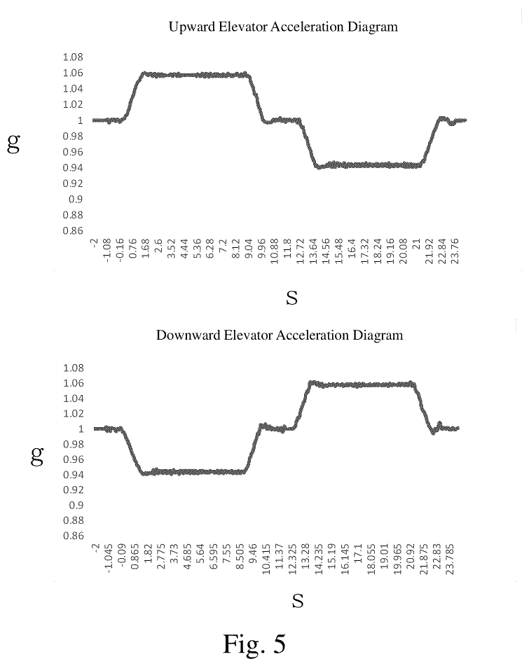

As shown in FIG. 1-FIG. 7, the present invention provides a method for analyzing the comfort level of high-speed elevator passengers based on EEG. It comprises the steps of:
S1: simultaneously collecting and storing human EEG signals of high-speed elevator passengers and operating signals of high-speed elevator cars;
S2: judging the changes in operating state of high-speed elevators based on operating signals of elevator cars; intercepting the human EEG signals and the operating signals of elevator cars during the changes in operating state, to obtain human EEG data and elevator car operating data;
S3: preprocessing human EEG data and elevator car operating data;
S4: conducting feature extraction for human EEG data and elevator car operating data, and forming human EEG index and elevator car operating index after analysis, wherein the step of conducting feature extraction for human EEG data includes the power spectrum analysis on intercepted EEG signals and the effective connection analysis on EEG signals;
S5: forming human EEG index curve and elevator car operating index curve by recording and storing human EEG index and elevator car operating index, wherein the index curve is the quantitative trajectory of single index mapped in two-dimensional Cartesian coordinate system.

Firstly, the experimenter places the acceleration sensor at the center of the high-speed elevator car to collect the acceleration signals of high-speed elevators. The sampling rate of the acceleration sensor is 1 kHz, and the resolution rate is 0.0005 g/LSB. The experimenter puts the 32-lead EEG electrode cap on the head of the passenger, the sampling rate of the EEG acquisition system is 1 kHz, the electrode distribution follows the 10-10 international standard lead system, the Cz electrode is set as the reference electrode, and the Fpz electrode is set as the ground electrode. Passengers need to clean their hair, and apply the conductive paste on the electrode position, to ensure that the electrode impedance is lower than 5 kΩ during EEG recording. The experiment sends synchronized timestamps to the EEG acquisition system and acceleration sensor in the form of serial communication, and the timestamp is sent once every 30 s.

Under the guidance of the experimenter, the passenger enters the elevator, and stands with eyes closed near the center of the elevator car. Collect acceleration signals of elevators in stationary state, as well as resting-state EEG signals of elevator passengers in stationary state. Extract three-axis acceleration signals of elevators, and use the Euclidean norm as the elevator acceleration data. Transmit the acceleration data to the trigger module. This module calculates the confidence interval of the acceleration data of elevators in stationary state. The calculation formula is as follows:

$$Q = \overline{X} \pm {Z}_{α / 2} \frac{σ}{\sqrt{n}}$$

Where, Z_{α/2} is the Z value at the 95% confidence level, and is approximately equal to 1.96; X̅ is the mean of vertical acceleration samples; σ is the standard deviation; n is the number of samples, and Q is the limit of the confidence interval. The acceptance domain scope of the data is obtained through the above calculation.

Then, under the operation of the experimenter, each passenger experiences four elevator trips: “upward-downward-upward-downward”. During the operation of the elevator, record acceleration signals of elevators and EEG signals of passengers. Use the start and end times of acceleration data segments that exceed the upper confidence limit or fall below the lower confidence limit, as elevator acceleration event window.

### Preprocess EEG Signals.

Filter EEG data with 0.5-45 Hz filter, and reduce the sampling signal to 250 Hz, to remove the baseline drift and power frequency interference of signals; then adjust the reference point of EEG signals to the mean reference; adopt the pseudo shadow space reconstruction algorithm to remove the bad data segments in signals; resample the human EEG data and the elevator car operating data; employ the independent component analysis algorithm to remove eye movement and electromyographic interference in EEG data; conduct the time synchronization for EEG and acceleration data based on synchronized timestamps of sensors; capture EEG data through the elevator acceleration event window;

### Conduct Power Spectrum Analysis on Intercepted EEG Signals.

Use the Welch algorithm to calculate the power spectrum density of each data segment in different EEG frequency bands (Delta frequency band: 1-4 Hz, Theta frequency band: 4-8 Hz, Alpha frequency band: 8-13 Hz, Beta frequency band: 13-30 Hz, Gamma frequency band: 30-45 Hz) Welch adopts the Hanning window to calculate the power spectrum density for each data segment of EEG sample, and obtain the mean of each port. Extract the effective connection feature of EEG data in frontal lobe region, and employ the direct directed transfer function to calculate the effective connection value of each EEG port; calculate the mean of effective connection values within each brain region, as well as the mean of the effective connection values among brain regions, as effective connection features.

### Carry Out the Effective Connection Analysis on EEG Signals.

The parameters of effective connection values are calculated based on the direct directed transfer function of Granger causality. The time-varying window analysis is used to establish the multivariate autoregressive model of the sliding window. The rectangular window is selected, with the duration of 0.4 s and the stepping of 0.03 s.

The multivariate autoregressive model is defined below:

$$X ( t ) = {\sum }_{n = 0}^{p} A ( n ) X ( t - n ) + E ( t )$$

Where, X(t)=[X₁(t), . . . , Xᵢ(t), . . . , X₃₂(t)]^{T} is the 32-channel EEG time series, A(n) is 32×32 coefficient matrix, E(t) is the remainder of white noise, and p is the order of the multivariate autoregressive model determined by the Hannan-Quinn information criterion.

The Fast Fourier Transform is used to convert the above formula into the frequency domain, as shown below:

$$X ( f ) = H ( f ) E ( f )$$

Where,

$$H ( f ) = {( {\sum }_{n = 0}^{p} A ( n ) {e}^{- i 2 π fn} )}^{- 1}$$

is the transfer matrix of the system, f is the frequency, and i is the complex number. The direct directed transfer function is defined below:

$${δ}_{ij}^{2} ( f ) = {η}_{ij}^{2} ( f ) {P}_{ij}^{2} ( f )$$

Where,

$${η}_{ij}^{2} ( f )$$

is the full-frequency directed transfer function; i and j are respectively the row and column numbers of the matrix.

$${η}_{ij}^{2} ( f ) = \frac{{\vert {H}_{ij} ( f ) \vert}^{2}}{{\sum }_{f} {\sum }_{m = 1}^{k} {\vert {H}_{im} ( f ) \vert}^{2}}$$

Where, k is the number of channels, and m is the column number of the full-frequency directed transfer function

$${η}_{ij}^{2} ( f ) .$$

Pᵢⱼ(ƒ) is the partial coherence function, defined below:

$${P}_{ij} ( f ) = \frac{{S}_{ij}^{- 1} ( f )}{\sqrt{{S}_{ii}^{- 1} ( f ) {S}_{jj}^{- 1} ( f )}}$$

Its expression is as follows:

$$S ( f ) = X ( f ) {X ( f )}^{*}$$

Where, S(ƒ) is the power spectrum function.

Calculate the stacking mean of the direct directed transfer function matrix of all data segments after obtaining the direct directed transfer function matrix of each data segment. Divide it into different frequency bands, and get the mean of the direct directed transfer function for different connection directions in each frequency band as an EEG effective connection strength index.

Finally, the statistical analysis is conducted for the resting-state human EEG data features and the EEG data features intercepted through the acceleration event window. The analysis results indicate that, under the influence of the high-speed elevator acceleration, the absolute power of the entire human brain's Theta band increases from 0.45±0.022 μV²/Hz to 0.726±0.197 μV²/Hz.

Table 1 shows the EEG characteristic parameters (mean±standard deviation) of baseline and acceleration states, as well as the corresponding results of p values for their comparison.

TABLE 1

| EEG characteristic | Human body in | Human body affected | p |
|---|---|---|---|
| parameters | resting state | by elevator acceleration | value |
| Theta absolute | 0.45 ± 0.022 | 0.726 ± 0.197 | <0.01 |
| power (μV²/Hz) | | | |

Based on the above analysis, compared with the existing technologies, the present invention achieves the human EEG data acquisition and analysis of high-speed elevator passengers, which serves as the basis for analyzing the comfort level.

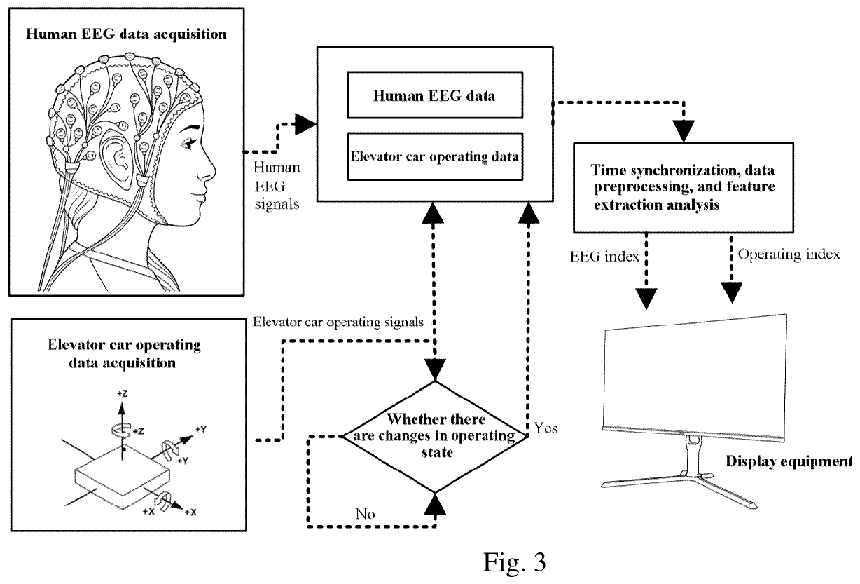

As shown in FIG. 3, the present invention also provides a system for analyzing the comfort level of high-speed elevator passengers based on EEG, and it includes an acquisition module, a trigger module, and a data analysis module. The acquisition module is used to collect human EEG signals of passengers and operating signals of high-speed elevator cars. The trigger module is coupled with the elevator car operating data acquisition module, configured to judge the changes in operating state of high-speed elevator cars based on elevator car operating signals, and output activation signals to data analysis module during the changes in operating state. The data analysis module is configured to receive activation signals, human EEG signals and elevator car operating signals; intercept and record human EEG signals and elevator car operating signals based on activation signals, to obtain human EEG data and elevator car operating data; perform time synchronization, data preprocessing and feature extraction analysis for human EEG data and elevator car operating data, to output EEG indexes and elevator car operating indexes. The acquisition module includes a human EEG data acquisition module, used to collect human EEG signals of passengers; an elevator car operating data acquisition module, used to collect high-speed elevator car operating signals;

When the trigger module judges the changes in operating state of high-speed elevator cars, the specific process includes: setting the threshold value according to the historical data of the elevator car operating signals; judging if the elevator car operating signals exceed the threshold range; outputting activation signals when the judgement result is “yes”; stopping outputting activation signals when the judgement result is “no”. The threshold value is set to be the 95% confidence interval of the statistical distribution of historical data.

When the acquisition module collects human EEG data, the EEG sensor is used for collection; the EEG sensor adopts an EEG electrode cap and sets it on the head of the passenger; according to the relevant regulations of GB/T 24474.1-2020 Measurement of Ride Quality—Party 1: Elevators, when the acquisition module collects elevator car operating data, the operation sensor is used for collection; the operation sensor adopts an acceleration sensor and sets it at the center of the bottom of the high-speed elevator car.

The operating index includes the operating data's time domain feature, frequency domain feature, and information entropy; the EEG index includes the EEG data's time domain feature, frequency domain feature, information entropy and effective connection.

The present invention also provides an intelligent terminal, including at least a processor and a machine-readable storage medium communicating and connecting with at least one processor, wherein the machine-readable storage medium has computer instructions executed by at least one processor, and the computer instructions are used to implement the system for analyzing the comfort level of high-speed elevator passengers based on EEG.

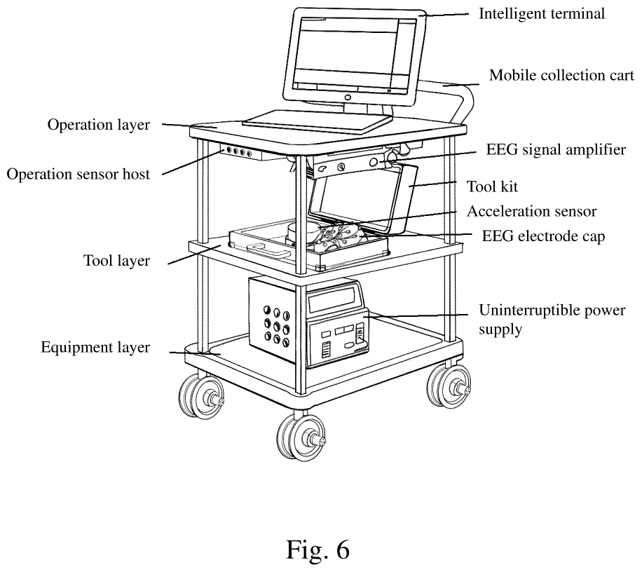

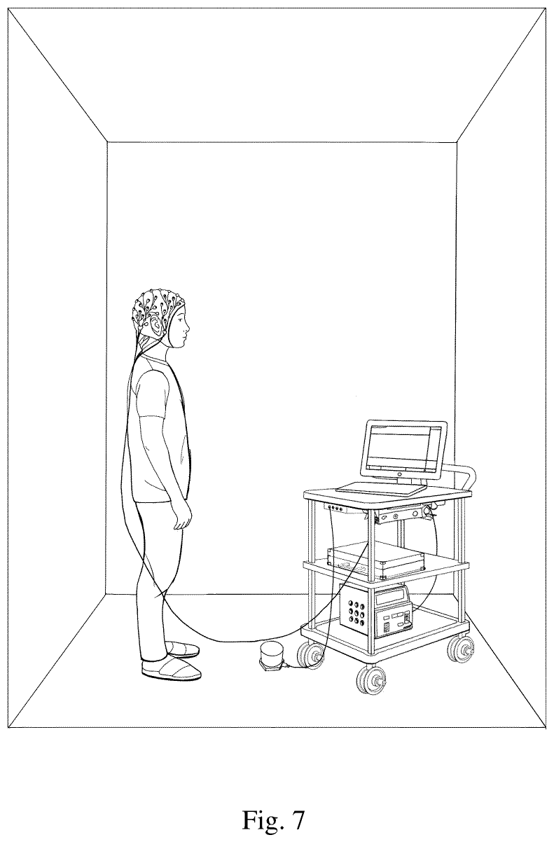

As shown in FIG. 6-FIG. 7, the present invention also provides a device for analyzing the comfort level of high-speed elevator passengers based on EEG, including:
an EEG sensor, an operation sensor, an intelligent terminal, an uninterruptible power supply, a tool kit, and a mobile collection cart; the EEG sensor includes an EEG electrode cap and an EEG signal amplifier; the operation sensor includes the acceleration sensor and operation sensor host; the mobile collection cart includes an equipment layer, a tool layer, and an operation layer; the equipment layer and the tool layer are rigidly connected to each other through four sets of fixed rods; the tool layer and the operation layer are rigidly connected to each other through four sets of fixed rods; the equipment layer includes a storage chassis and a universal wheel connected to the bottom of the storage chassis, used to place the uninterruptible power supply; the tool layer is used to place the acceleration sensor, the EEG electrode cap, and the tool kit; the operation layer includes an operation table, wherein a handrail is set up on one side of the operation table at least, and one side of the bottom of the operation table is detachably connected to the EEG signal amplifier and the operation sensor host; the operation layer is used to place the intelligent terminal; the EEG sensor, the operation sensor, the uninterruptible power supply, and the intelligent terminal are electrically connected to each other;
During the analysis process, the human EEG data are collected through the EEG sensor, and the elevator car operating data are acquired through the operation sensor; the collected data are input into an intelligent terminal, and the intelligent terminal analyzes and judges the collected data, and ultimately outputs human EEG index and elevator car operating index. The specific analysis and judgement process of the collected data by the intelligent terminal is the same as the process in the method for analyzing the comfort level of high-speed elevator passengers based on EEG, which is not provided here.

Obviously, the described embodiments are only a part of the embodiments of the present invention, not all of them. Based on the embodiments of the present invention, other embodiments obtained by the general technicians in the field without creative design fall within the protection scope of the present invention.

What is claimed is:

1. A method for analyzing the comfort level of high-speed elevator passengers based on EEG, wherein it comprises the steps of:
S1: simultaneously collecting and storing human EEG signals of high-speed elevator passengers and operating signals of high-speed elevator cars;
S2: judging the changes in operating state of high-speed elevators based on operating signals of elevator cars; intercepting the human EEG signals and the operating signals of elevator cars during the changes in operating state, to obtain human EEG data and elevator car operating data;
the step of intercepting human EEG data and elevator operating data includes:
collecting acceleration signals of elevators in stationary state, as well as resting-state EEG signals of elevator passengers in stationary state; extracting three-axis acceleration signals of elevators, and using the Euclidean norm as the elevator acceleration data;
calculating the confidence interval of the acceleration data of the elevator in stationary state;
recording acceleration signals of elevator cars and EEG signals of passengers during the operation of elevators;
using the start and end times of acceleration data segments that exceed the upper confidence limit or fall below the lower confidence limit, as elevator acceleration event window;
S3: preprocessing the human EEG data and elevator car operating data;
the preprocessing step includes:
filtering EEG data with 0.5-45 Hz filter, and reducing the sampling signal to 250 Hz, to remove the noise in the human EEG data;
adopting the pseudo shadow space reconstruction algorithm to remove the bad data segments in human EEG data;
employing the independent component analysis algorithm to remove the interference signals in the human EEG data;
conducting the time synchronization for EEG and acceleration data based on synchronized timestamps;
capturing EEG data through the elevator acceleration event window;
S4: conducting the feature extraction for the human EEG data and elevator car operating data, and forming human EEG index and elevator car operating index after analysis, wherein the step of conducting feature analysis for human EEG data includes the power spectrum analysis on intercepted EEG signals and the effective connection analysis on EEG signals;
the step of conducting power spectrum analysis on intercepted EEG signals includes: using the Welch algorithm to calculate the power spectrum density of each data segment in different EEG frequency bands as an EEG power spectrum index, wherein the EEG frequency bands include Delta frequency band, Theta frequency band, Alpha frequency band, Beta frequency band, and Gamma frequency band, and the Welch algorithm adopts the Hanning window to calculate the power spectrum density for each data segment of EEG sample, and obtain the mean of each EEG signal port; extracting the effective connection feature of EEG data, and employing the direct directed transfer function based on Granger causality to calculate the effective connection value of each EEG port; calculating the mean of effective connection values within each brain region, as well as the mean of the effective connection values among brain regions, as effective connection features;
S5: forming human EEG index curve and elevator car operating index curve by recording and storing the human EEG index and elevator car operating index, wherein the index curve is the quantitative trajectory of single index mapped in two-dimensional Cartesian coordinate system.

2. The method for analyzing the comfort level of high-speed elevator passengers based on EEG described in claim 1, wherein, in S4, the specific step of conducting the effective connection analysis on EEG signals includes: obtaining the direct directed transfer function matrix of each data segment; calculating the mean of the direct directed transfer function matrix for all data segments, dividing it into different frequency bands, and getting the stacking mean of the direct directed transfer function for different connection directions in each frequency band as an EEG effective connection index.

3. A system for analyzing the comfort level of high-speed elevator passengers based on EEG, wherein it includes:
an acquisition module, used to collect human EEG signals of passengers and operating signals of high-speed elevator cars;
a trigger module, coupled with the elevator car operating signal acquisition module, configured to judge the changes in operating state of high-speed elevator cars based on the elevator car operating signals, and output activation signals to data analysis module during the changes in operating state;
a data analysis module, configured to receive the activation signals, the human EEG signals and the elevator car operating signals; intercept and record the human EEG signals and the elevator car operating signals based on the activation signals, to obtain human EEG data and elevator car operating data;
the step of intercepting EEG data includes:
collecting acceleration signals of elevators in stationary state, as well as resting-state EEG signals of elevator passengers in stationary state; extracting three-axis acceleration signals of elevators, and using the Euclidean norm as the elevator acceleration data;
calculating the confidence interval of the acceleration data of the elevator in stationary state;
recording acceleration signals of elevators and EEG signals of passengers during the operation of elevators;
using the start and end times of acceleration data segments that exceed the upper confidence limit or fall below the lower confidence limit, as elevator acceleration event window;
performing time synchronization, data preprocessing and feature extraction analysis for the human EEG data and elevator car operating data, to output EEG indexes and elevator car operating indexes;
the preprocessing step includes:
filtering EEG data with 0.5-45 Hz filter, and reducing the sampling signal to 250 Hz, to remove the noise in the human EEG data;
adopting the pseudo shadow space reconstruction algorithm to remove the bad data segments in the signals;
employing the independent component analysis algorithm to remove the interference signals in the human EEG data;
conducting the time synchronization for EEG and acceleration data based on synchronized timestamps of sensors;
capturing EEG data through the elevator acceleration event window;
conducting power spectrum analysis on intercepted EEG signals by using the Welch algorithm at the time of carrying out the feature analysis on human EEG data to obtain the power spectrum index of EEG data, and implementing the effective connection analysis on EEG signals through the direct directed transfer function to get the effective connection index of EEG data;
the step of conducting power spectrum analysis on intercepted EEG signals includes: using the Welch algorithm to calculate the power spectrum density of each data segment in different EEG frequency bands as an EEG power spectrum index, wherein the EEG frequency bands include Delta frequency band, Theta frequency band, Alpha frequency band, Beta frequency band, and Gamma frequency band, and the Welch algorithm adopts the Hanning window to calculate the power spectrum density for each data segment of EEG sample, and obtain the mean of each EEG signal port; extracting the effective connection feature of EEG data, and employing the direct directed transfer function based on Granger causality to calculate the effective connection value of each EEG port; calculating the mean of effective connection values within each brain region, as well as the mean of the effective connection values among brain regions, as effective connection features.

4. The system for analyzing the comfort level of high-speed elevator passengers based on EEG described in claim 3, wherein, when the trigger module judges the changes in operating state of high-speed elevator cars, the process includes:
setting the threshold value according to the historical data of the elevator car operating signals, judging if the elevator car operating signals exceed the threshold range, outputting activation signals when the judgement result is “yes”, and stopping outputting activation signals when the judgement result is “no”.

5. The system for analyzing the comfort level of high-speed elevator passengers based on EEG described in claim 3, wherein the elevator car operating signals include acceleration signals, speed signals, and attitude signals; the elevator car operating index includes the operating data's time domain feature, frequency domain feature, and information entropy; the EEG index includes the EEG data's time domain feature, frequency domain feature, information entropy and effective connection.

6. A device for analyzing the comfort level of high-speed elevator passengers based on EEG, used to conduct the analysis for the method for analyzing the comfort level of high-speed elevator passengers based on EEG described in claim 1, wherein it includes:
an EEG sensor, an operation sensor, an intelligent terminal, an uninterruptible power supply, a tool kit, and a mobile collection cart; the EEG sensor includes an EEG electrode cap and an EEG signal amplifier; the operation sensor includes the acceleration sensor and operation sensor host; the mobile collection cart includes an equipment layer, a tool layer, and an operation layer from top to bottom, and these layers are rigidly connected to each other through four sets of fixed rods; the equipment layer includes a storage chassis and a universal wheel connected to the bottom of the storage chassis, used to place the uninterruptible power supply; the tool layer is used to place the acceleration sensor, the EEG electrode cap, and the tool kit; the operation layer includes an operation table, wherein a handrail is set up on one side of the operation table, and one side of the bottom of the operation table is detachably connected to the EEG signal amplifier and the operation sensor host; the operation layer is used to place the intelligent terminal; the EEG sensor, the operation sensor, the uninterruptible power supply, and the intelligent terminal are electrically connected to each other;
during the analysis process, human EEG data are collected through the EEG sensor, and elevator car operating data are acquired through the operation sensor; the collected data are input into an intelligent terminal to judge the changes in the operating state of the elevator car, preprocess human EEG data and elevator car operating data, perform feature extraction analysis, and ultimately output human EEG index and elevator car operating index.

7. A device for analyzing the comfort level of high-speed elevator passengers based on EEG, used to conduct the analysis for the method for analyzing the comfort level of high-speed elevator passengers based on EEG described in claim 2, wherein it includes:
an EEG sensor, an operation sensor, an intelligent terminal, an uninterruptible power supply, a tool kit, and a mobile collection cart; the EEG sensor includes an EEG electrode cap and an EEG signal amplifier; the operation sensor includes the acceleration sensor and operation sensor host; the mobile collection cart includes an equipment layer, a tool layer, and an operation layer from top to bottom, and these layers are rigidly connected to each other through four sets of fixed rods; the equipment layer includes a storage chassis and a universal wheel connected to the bottom of the storage chassis, used to place the uninterruptible power supply; the tool layer is used to place the acceleration sensor, the EEG electrode cap, and the tool kit; the operation layer includes an operation table, wherein a handrail is set up on one side of the operation table, and one side of the bottom of the operation table is detachably connected to the EEG signal amplifier and the operation sensor host; the operation layer is used to place the intelligent terminal; the EEG sensor, the operation sensor, the uninterruptible power supply, and the intelligent terminal are electrically connected to each other;
during the analysis process, human EEG data are collected through the EEG sensor, and elevator car operating data are acquired through the operation sensor; the collected data are input into an intelligent terminal to judge the changes in the operating state of the elevator car, preprocess human EEG data and elevator car operating data, perform feature extraction analysis, and ultimately output human EEG index and elevator car operating index.

8. An intelligent terminal, wherein it includes:
at least a processor, and a machine-readable storage medium communicating and connecting with at least one processor; the machine-readable storage medium has computer instructions executed by at least one processor; the computer instructions are used to implement the method for analyzing the comfort level of high-speed elevator passengers based on EEG described in claim 1.

9. An intelligent terminal, wherein it includes:
at least a processor, and a machine-readable storage medium communicating and connecting with at least one processor; the machine-readable storage medium has computer instructions executed by at least one processor; the computer instructions are used to implement the method for analyzing the comfort level of high-speed elevator passengers based on EEG described in claim 2.
